(19) United States
(12) Patent Application Publication
(10) Pub. No.: US 2009/0124869 A1
(43) Pub. Date: May 14, 2009

(54) Medical Apparatus Capable of Recording Physiological Signals

(76) Inventors: Wei-Chih HU, Chung Li (TW); Liang-Yu Shyu, Chung Li (TW); Yiu-Tong Chu, Panchiao City (TW); Ai-Hsien Li, Taipei City (TW); Jenn-Lung Su, Chung Li (TW); Yuh-Show Tsai, Chung Li (TW); Shaou-Gang Miaou, Chung Li (TW); Shao-Tien Chen, Chung Li (TW)

Correspondence Address: SINORICA, LLC, 528 FALLSGROVE DRIVE, ROCKVILLE, MD, 20850, US

(21) Appl. No.: 12/116,982

(22) Filed: May 8, 2008

(30) Foreign Application Priority Data
Nov. 8, 2007 (TW) 096142272

(51) Int. Cl. A61B 5/0476; A61B 5/00; A61B 5/0432

(52) U.S. Cl. 600/301; 600/523; 600/544

(57) ABSTRACT

The present invention related to a medical apparatus capable of recording physiological signals, which uses a EEG detecting circuit to detect the human brain and produce a EEG signal, a ECG detecting circuit to detect the human heart and produce a ECG signal, a microcontroller circuit to receive the EEG and ECG signals and produce a control signal, and a storage unit to receive the control signal and store the EEG and ECG signals. The EEG and ECG signals are correlated. Accordingly, physicians can analyze diseases conveniently.

### FIELD OF THE INVENTION

The present invention relates generally to a medical apparatus, and particularly to a medical apparatus capable of recording physiological signals.

### BACKGROUND OF THE INVENTION

In recent years, people think highly of the cerebrovascular and heart diseases, which are major causes of death only next to malignancies. Presently, the morbidity of cerebrovascular diseases is increasing yearly, and the heart diseases, diabetes, and hypertension are the major accomplices of cerebral apoplexy. Diseases with such high danger are worth our emphasis and attention.

The coronary artery disease is one of the common chronic diseases at the present day. The morbidity thereof is only next to the hypertension and cerebral apoplexy. In many publications, it is reported that the coronary artery disease accompanies the cerebral vessel stenosis, which causes post-Coronary Artery Bypass Grafting (CABG) or post-Percutaneous Transluminal Coronary Angioplasty (PTCA) complications including cerebral apoplexy or long-term intellectual deterioration. The blood flow barrier in brain scans of the nuclear medicine proved to be relevant to such complications. In addition, after the coronary artery operation, patients usually suffer from intellectual deterioration and other neural complications, which are usually caused by microinfarcts induced by arrhythmia, and particularly atrial fibrillation, in coronary heart diseases. The authors of the present invention believe that another significant reason, which is usually ignored, is that patients with the coronary artery disease suffer from the occlusive cerebrovascular disease.

When the organic pathological changes or functional disorder occur in the brain of a patient with the cerebrovascular diseases, electrical physiology of neural cells will be influenced, resulting in different variations in electroencephalogram. When the pathological changes occur in the brain, neural cells necrose and lose the original electrical activities. In large-scale cerebral infarction, the brain waves will disappear or show decrease in amplitude. The half-period pathological change region in the brain and its surrounding region appear regional slow waves of θ and δ. Normally, the poly delta activity (PDA) appears. The regional slow waves can possibly replace the original normal brain waves, or combine with the original background brain waves. The variations of brain waves of patients with cerebral apoplexy include poly large-amplitude slow waves, loss of fundamental background waves, and intermittent periodic δ waves. Epilepsy-like brain waves appear in some patients with acute cerebral apoplexy. When clots exist in the brain, flat brain waves with significantly reduced amplitude appear. Cerebral vessel stenosis causes anoxic phenomenon in brains. The variations in electroencephalogram depend on the degree and duration of anoxaemia. In most severe cases, the α-coma, the burst-suppression pattern, or the flat EEG will appear.

The heart functions like a pump. When patients with cardiovascular diseases suffer from valve destruction caused by any nidus and the load of the heart is increased, the symptoms of heart diseases occur. The reasons of the load increase of the heart include the resistance caused by stenosis and leaky valve, which is valvular insufficiency and causes backflow of blood between the atrium and the ventricle. Once the load is increased, symptoms of heart failure occur. Depending on the degree of stenosis or valvular insufficiency, different degrees of symptoms appear. When any of the valves has a problem, the ventricle will expand or become blubber. Consequently, the load of the heart is increased, and the heart thereby cannot supply blood with high oxygen content to the whole body. Then the blood to the brain or to the lower limbs will be insufficient, and the anoxia will occur. The light anoxia causes increases in the heart rate, cardiac output, and blood pressure. More severely, when serious anoxia occurs, the blood pressure, heart rate, and the cardiac output will decrease, resulting in arrhythmia, ventricular fibrillation, and cardiac arrest. Thereby, when the stenosis happens, the heart rate in the electrocardiogram varies depending on the degree of anoxia, and the electroencephalogram exhibits abnormal situation owing to anoxic condition in the feet.

When the blood flow in the heart is insufficient, the blood transported to the brain is insufficient as well, resulting in anoxic condition in the brain. The degree and the duration of which anoxic condition in the brain influence the brain waves. Most seriously, α-com, burst-suppression brain wave, or flat EEG will happen. When the arrhythmia occurs, the thrombus forms in the blood. When the clots in the blood enter the brain, cerebrovascular embolism will result, causing regional slow brain waves.

When the cerebral ischemia or cerebral hemorrhage occurs, the brain can adjust the activities of the autonomic nerve system to speed up the heart rate and the respiration as well as to increase pressure in the heart for conveying more oxygen. Thereby, the oxygen content in the brain can recover balance.

In order to clarify the disease history and danger factors of such kind of patients, for the patients with the coronary artery disease and the cerebral vessel stenosis (the first group) and for the patients preparing to accept operation or Percutaneous Transluminal Coronary Angioplasty (the second group), a 24-hour portable simultaneous recorder for the electrocardiogram (ECG) and electroencephalogram (EEG) is designed and manufactured for analyzing the acquired signals and then understanding their correlations. The primary goal is the timing correlation between EEG and ECG. Afterwards, according to the correlation, the regression models of all relevant coefficients are constructed. It is believed that such models not only can explain the neural complications after operations as described above, but also can be a significant foundation for such complications. In addition, for ischemic apoplexy (the third group), it is regarded for a long time to be related to paroxysmal arrhythmia, particularly atrial fibrillation, and intracardiac thrombus. Though such paroxysmal atrial fibrillation cannot be detected by regular ECG, it can result in ischemic apoplexy. It is hoped that such a 24-hour portable simultaneous recorder for the ECG and EEG can clarify the problems.

Accordingly, the present invention provides a novel medical apparatus capable of recording physiological signals, which can record simultaneously ECG and EEG accurately and display the ECG and EEG signals in a the display of a computer for examination. The physicians thereby can analyze the correlation of the signals.

### SUMMARY

An objective of the present invention is to provide a medical apparatus capable of recording physiological signals, which can detect and record the ECG and EEG signals of a human body simultaneously. Thereby, physicians can analyze diseases conveniently.

Another objective of the present invention is to provide a medical apparatus capable of recording physiological signals, which can analyze the correlation of the ECG and EEG signals of a human body. Thereby, physicians can analyze diseases conveniently.

The medical apparatus capable of recording physiological signals according to the present invention comprises an EEG detecting circuit, an ECG detecting circuit, a microcontroller circuit, and a storage unit. The EEG detecting circuit detects the human brain and produces an EEG signal. The ECG detecting circuit detects the human heart and produces an ECG signal. The microcontroller circuit receives the EEG and ECG signals, and produces a control signal. The storage unit stores the EEG and ECG signals according to the control signal. The ECG signal correlates to the EEG signal.

Besides, the medical apparatus capable of recording physiological signals according to the present invention further comprises an analysis unit, which receives and analyzes the EEG and ECG signals according to the control signal, and then produces an analysis signal for physicians for analyzing diseases.

### BRIEF DESCRIPTION OF THE DRAWINGS

FIG. 1 shows a block diagram according to a preferred embodiment of the present invention;

FIG. 2 shows a block diagram of the EEG detecting circuit according to a preferred embodiment of the present invention;

FIG. 3 shows a schematic diagram of the locations to set EEG electrodes according to a preferred embodiment of the present invention;

FIG. 4 shows a schematic diagram of the locations to set ECG electrodes according to a preferred embodiment of the present invention;

FIG. 5 shows a schematic diagram of the display interface of the display device according to a preferred embodiment of the present invention;

FIG. 6 shows a schematic diagram of the keying functions of the input unit according to a preferred embodiment of the present invention;

FIG. 7 shows a block diagram of the analysis unit according to a preferred embodiment of the present invention; and

FIG. 8 shows a flowchart of analyzing the ECG and EEG signals by the analysis unit according to a preferred embodiment of the present invention.

### DETAILED DESCRIPTION

In order to make the structure and characteristics as well as the effectiveness of the present invention to be further understood and recognized, the detailed description of the present invention is provided as follows along with preferred embodiments and accompanying figures.

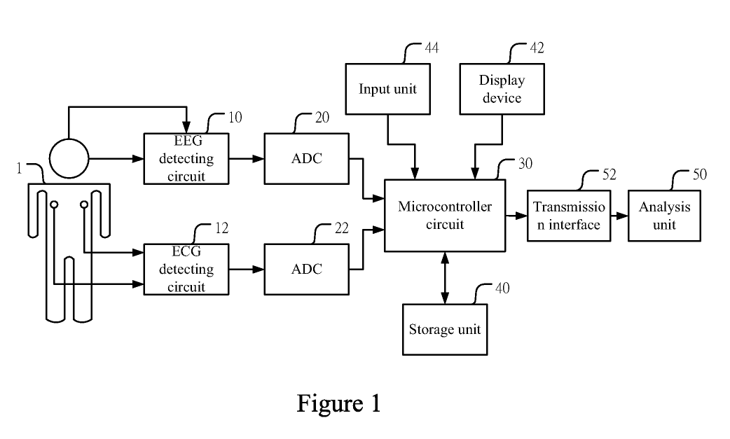

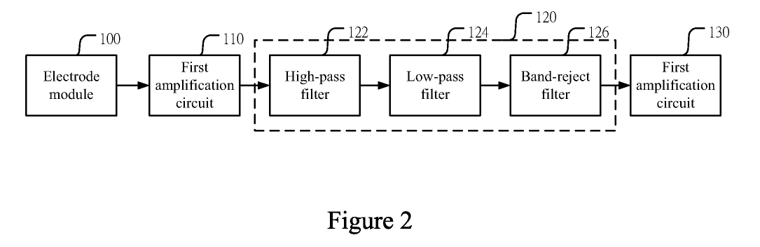

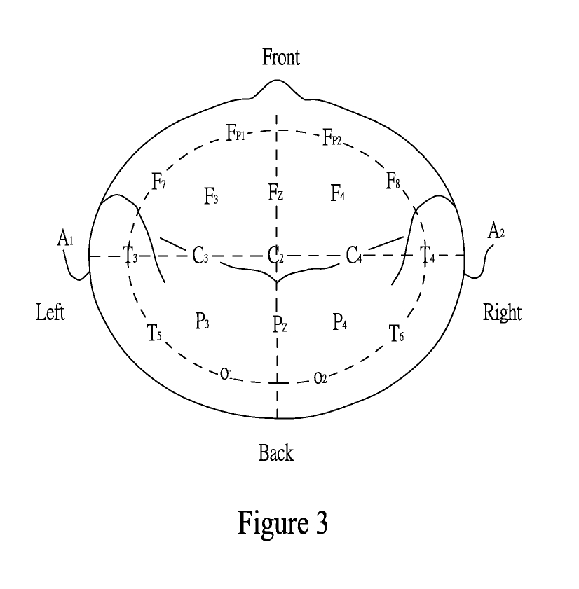

FIG. 1 shows a block diagram according to a preferred embodiment of the present invention. As shown in the figure, the medical apparatus capable of recording physiological signals according to the present invention comprises an EEG detecting circuit 10, an ECG detecting circuit 12, analog-to-digital converter circuits 20, 22, a microcontroller circuit 30, and a storage unit 40. The EEG detecting circuit 10 is used for detecting the human brain and produces an EEG signal. FIG. 2 shows a block diagram of the EEG detecting circuit according to a preferred embodiment of the present invention. As shown in the figure, the EEG detecting circuit 10 comprises an electrode module 100, a first amplification circuit 110, a filter circuit 120, and a second amplification circuit 130. The electrode module 100 comprises six electrodes, distributed uniformly over the human head (as shown in FIG. 3). Namely, they are distributed over the six points Fp1, Fp2, F3, F4, C3, and C4 on the head. In addition, they are divided into two sides. The left side uses the points Fp1, F3, and C3 as the positive terminal (Vin+), and the point A1 as the negative terminal (Vin−); the right side uses the points Fp2, F4, and C4 as the positive terminal (Vin+), and the point A2 as the negative terminal (Vin−). The chin is used as the reference ground. Thereby, the EEG signal can be detected uniformly for understanding thoroughly the condition of each part of the brain.

The first amplification circuit 110 is an instrumentation amplifier. Because the EEG signal is very weak and unstable, it is difficult to measure the signal. Thereby, the first amplification circuit 110 receives the EEG signal detected by the electrode module 100 for amplifying the weak physiological signal, namely, the EEG signal. The filter circuit 120 receives the EEG signal amplified by the first amplification circuit 110 and filters the noises therein. The filter circuit 120 further includes a high-pass filter 122, a low-pass filter 124, and a band-reject filter 126. The high-pass filter 122 receives the EEG signal amplified by the first amplification circuit 110, and filters the low-frequency drift components in the EEG signal. Thereby, low-frequency interference during measurement can be avoided. The high-pass filter 122 is a Butterworth high-pass filter. In order to retain the most of the ECG signal and eliminate unnecessary high-frequency noises, the low-pass filter 124 is set to receive and filter the high-frequency component of the ECG signal filtered by the high-pass filter 122. Thereby, the low-frequency drift component of the ECG signal can be filtered and the high-frequency interference, which is mainly 60 Hz appliance noises, during measurement can be prevented. The frequency range of the ECG signal is approximately between 1 and 30 Hz, thereby the cutoff frequency is set at 30 Hz. Meanwhile, the 60 Hz signal is filtered for the first time. It can be regarded as a pre-filter of the 60 Hz signal. The low-pass filter 124 is a fourth-order Butterworth low-pass filter. The band-reject filter 126 filters a noise frequency of the ECG signal filtered by the low-pass filter 122 for filtering the 60 Hz power-supply noise. The second amplification circuit 130 receives the ECG signal filtered by the filter circuit 120, and amplifies the ECG signal.

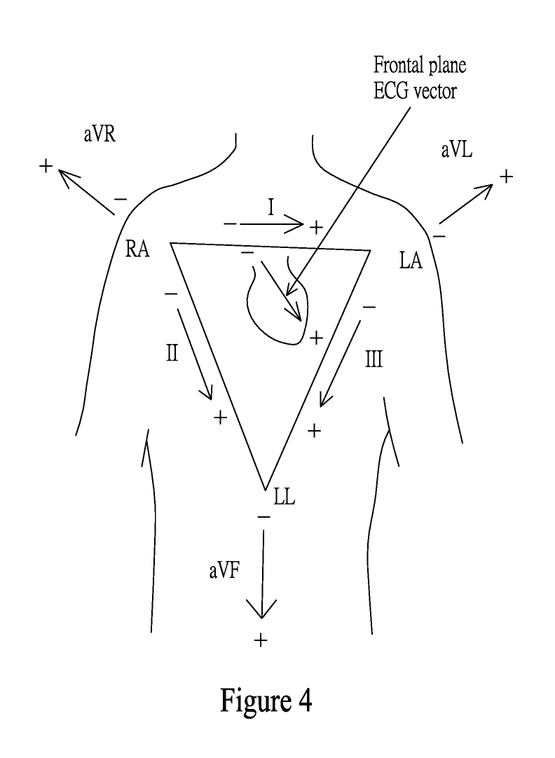

The ECG detecting circuit 12 detects the human heart and produces an ECG signal. The electrodes of the ECG detecting circuit 12 are placed on the RA and LA points (as shown in FIG. 4) of a human body, and the chin is used as the reference ground, just like the ECG detecting circuit 10. Because the circuit principle of the ECG detecting circuit 12 is the same the EEG detecting circuit 10, the details are not described further.

The analog-to-digital converter circuits 20, 22 receive the EEG and ECG signals, respectively, and convert the analog EEG and ECG signals to digital signals, respectively, and transmit to the microcontroller circuit 30. The microcontroller circuit 30 receives the digital EEG and ECG signals and produces a control signal. The storage unit 40 receives the control signal, and stores and records the EEG and ECG signals. Besides, because the size of the medical apparatus according to the present invention is small, it is convenient to carry while measuring and recording the EEG and ECG signals simultaneously.

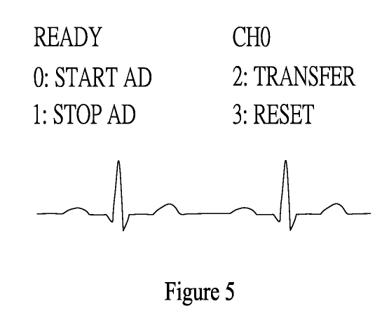

In addition, the medical apparatus capable of recording physiological signals further comprises a display device 42 and an input unit 44. The display device 42 is coupled to the microcontroller circuit 30, and receives and displays real-timely the ECG and EEG signals. The users can select the physiological signal to be displayed. At the upper left corner of the screen of the display device 42, the status of the present medical apparatus is displayed, telling the users that the present display device 42 is standing by, transmitting data, or extracting signals (as shown in FIG. 5). The display device 42 is a liquid crystal display (LCD).

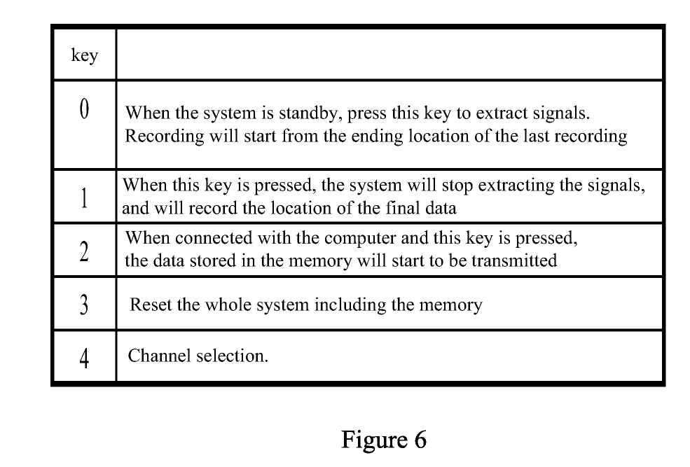

Moreover, in the interface menu of the display device 42, the users can use the input unit 44 for system operation. The input unit 44 is coupled to the microcontroller circuit 30, and used for transmitting an input signal and controlling the actions of the microcontroller circuit 30. The input unit 44 is a keyboard device, used for providing a communication interface between the users and the medical apparatus. Thereby, the users can control the medical apparatus by inputting through the keyboard with ease. As shown in FIG. 6, the functions of the keyboard device include: 0: Start to extract signals; 1: Stop extracting signals; 2: transmission; 3: System reset; and 4: Select channel.

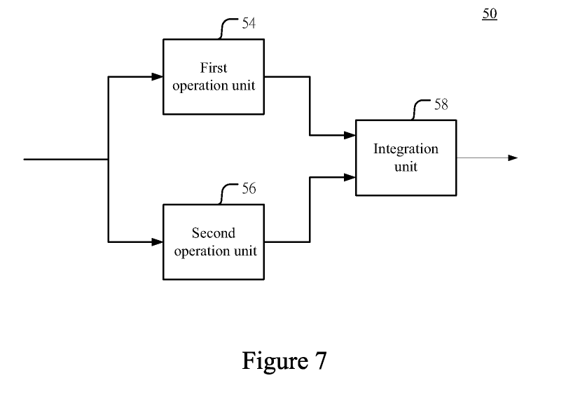

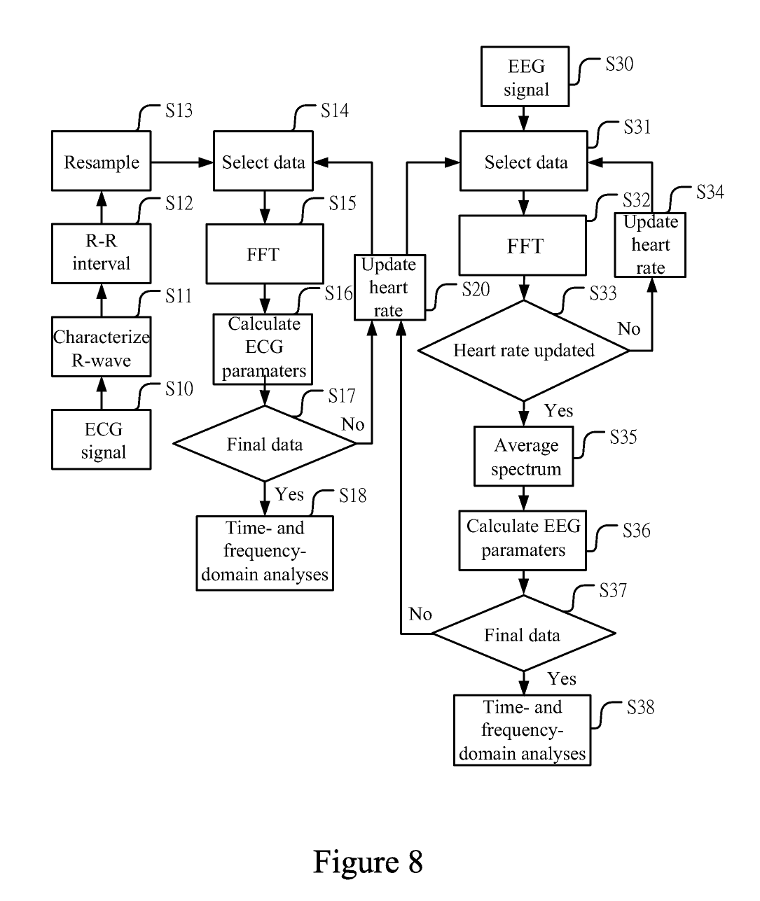

The analysis unit 50 receives the EEG and ECG signals according to the control signal, analyzes the correlation therebetween, and produces an analysis signal for convenient analysis of diseases for physicians. According to a preferred embodiment of the present invention, FIG. 7 shows a block diagram of the analysis unit, and FIG. 8 shows a flowchart of analyzing the ECG and EEG signals by the analysis unit. As shown in the figures, the analysis unit 50 according to the present invention includes a first operation unit 54, a second operation unit 56, and an integration unit 58. The first operation unit 54 receives and performs operations on the ECG signal, and then produces ECG parameters including α activity, β activity, δ activity, and θ activity. In the following, how the first operation unit 54 performs operations on the ECG signal to give the ECG parameters is described with the reference to FIG. 8. First, after the first operation unit 54 receives the ECG signal (the step S10), the ECG signal is characterized (the step S1) with the aim of searching the location of R-wave in the ECG signal. Then, the time of the R-R interval is calculated (as the step S12). After resampling in the step S13, by the step S15, fast Fourier transform (FFT) is used for giving the ECG parameters, which are parameters in heart-rate variations. Next, compare and observe using time synchronization and the spectrum components of EEG (as the step S18). According to the preferred embodiment of the present invention, 2048-point FFT is adopted (as the step S14), thereby, 2048 sample points have to be collected first before the power spectrum can be calculated. After calculating the power spectrum of heart-rate variations, in the power spectrum, several wave peaks can be found between the frequency range of 0 and 0.4 Hz. According to the present invention, the energy in the frequency band between 0.04 and 0.15 Hz is defined as low frequency power (LFP); the energy in the frequency band between 0.15 and 0.4 Hz is defined as high frequency power (HFP); and the frequency band between 0 and 0.5 Hz is defined as TP_hrv (hrv total power).

The second operation unit 54 performs operations on the EEG signal, and produces one or more EEG parameters. That is, the second operation unit 54 uses the original signal of the EEG and, the same as the first operation unit 52, adopts 2048 points (as the step S31). After FFT (as the step S32) to give the power spectrum, move backwards by 40 points and take a fixed length to give the next data. Thereby, 0.2-second time resolution is achieved. This procedure is continued for 30 heart rates (as the step S20). Then, average the power spectrum (as the step S35) and calculate the EEG parameters. The energy between the band 0.4 and 4 Hz is defined as δ activity; the energy between the band 4 and 8 Hz is defined as θ activity; the energy between the band 8 and 12 Hz is defined as α activity; the energy between the band 12 and 30 Hz is defined as β activity; and the energy between the band 0 and 30 Hz is defined as TP_eeg (EEG total power). For every 30 heart rates, a set of parameter data, including R-R interval, heart-rate-variation-related (HRV-related) LFP, HFP, and TP_hrv, and EEG-related δ, θ, α, β activities and TP_eeg, is calculated. Repeat the steps described above and update for every 30 heart rates, then the time- and frequency-domain power spectrum diagrams are given. The integration unit 58 receives and integrates the ECG and EEG parameters, and produces one or more relevant parameters. Afterwards, Pearson's correlation is used to find the Pearson's correlation coefficients, which are isotropic and ranged between −1.0 and 1.0, and are used for revealing the linear correlation between two sets of data.

The correlation between the β activity and R-R interval of sleep of a normal person is −0.749; and the correlation between the δ activity and LFP of sleep of a normal person is −0.477. Although the correlation between δ activity and LFP is only −0.447, the trend exhibits significant negative correlation. Thereby, in the present preferred embodiment, δ activity and LFP are used to perform event correlation analysis. An event threshold value is set to the respective parameter. The threshold value of δ activity is set as 0.20; the threshold value of LFP is set as 0.06. The parameters are compared to the threshold values. If the parameter is greater than the threshold value, then 1 is set; if not, 0 is given. This means that if the parameter is greater than the threshold value, δ activity event occurs, and LFP is active. After calculation, correlation analysis is performed, and the Pearson's correlation coefficient is given as −0.608, which shows high negative correlation. It could be because the sympathetic nerve is suppressed during deep sleep, and will be activated during REM sleep. Thereby, by the correlation between ECG and EEG parameters for a normal person, physicians can analyze abnormal condition of a human body.

Furthermore, the medical apparatus according to the present invention is a portable medical apparatus, which can record continuously the EEG and ECG signals of a human body and then transmit the signals to the analysis unit 50 via a transmission interface 52 coupled between the microcontroller circuit 30 and the analysis unit 50. The analysis unit can be set in a computer system for analyzing the correlation between the EEG and ECG signal. Hence, by recording the EEG and ECG signals of a human body in the medical apparatus according to the present invention for a long time, a user can record conveniently at home, and then the recorded data can be sent to the physicians for further analysis and judgment of diseases. The transmission interface 52 includes Peripheral Component Interconnect (PCI), Universal Serial Bus (USB), 1394, IEEE802.3 interface, IrDA, or Bluetooth interfaces. The interfaces described are only examples of interfaces according to a preferred embodiment of the present invention. The transmission interface of the present invention is not limited to the examples described above.

To sum up, the medical apparatus capable of recording physiological signals uses a EEG detecting circuit and a ECG detecting circuit to detect EEG and ECG signal of a human body simultaneously. In addition, an analysis unit is used for analyzing the correlation between the EEG and ECG signal. Accordingly, physicians can analyze diseases conveniently.

Accordingly, the present invention conforms to the legal requirements owing to its novelty, non-obviousness, and utility. However, the foregoing description is only a preferred embodiment of the present invention, not used to limit the scope and range of the present invention. Those equivalent changes or modifications made according to the shape, structure, feature, or spirit described in the claims of the present invention are included in the appended claims of the present invention.

1. A medical apparatus capable of recording physiological signals, comprising:
an EEG detecting circuit, detecting the human brain and producing an EEG signal;
an ECG detecting circuit, detecting the human heart and producing an ECG signal;
a microcontroller circuit, receiving the EEG and ECG signals, and producing a control signal; and
a storage signal, receiving the control signal, and storing the EEG and ECG signals, which are correlated.

2. The medical apparatus of claim 1, and further comprising an analysis unit, receiving the EEG and ECG signals according to the control signal, analyzing the EEG and ECG signals, and producing an analysis signal.

3. The medical apparatus of claim 2, and further comprising a transmission interface, coupled between the microcontroller circuit and the analysis unit, transmitting the EEG and ECG signal to the analysis unit.

4. The medical apparatus of claim 3, wherein the transmission interface includes Peripheral Component Interconnect (PCI), Universal Serial Bus (USB), 1394, IEEE802.3 interface, IrDA, or Bluetooth interfaces.

5. The medical apparatus of claim 3, wherein the analysis unit further includes:
a first operation unit, performing operations on the ECG signal, and producing one or more ECG parameter;
a second operation unit, performing operations on the EEG signal, and producing one or more EEG parameter; and
an integration unit, receiving and integrating the ECG and EEG parameters, and producing one or more relevant parameters.

6. The medical apparatus of claim 5, wherein the ECG parameters includes total power (TP), low frequency power (LFP), and high frequency power (HFP).

7. The medical apparatus of claim 5, wherein the EEG parameters includes α activity, β activity, δ activity, and θ activity.

8. The medical apparatus of claim 1, and further comprising an analog-to-digital converter, converting the analog EEG signal to a digital signal, and transmitting to the microcontroller circuit.

9. The medical apparatus of claim 1, and further comprising an analog-to-digital converter, converting the analog ECG signal to a digital signal, and transmitting to the microcontroller circuit.

10. The medical apparatus of claim 1, and further comprising an input unit, coupled to the microcontroller circuit, and transmitting an input signal for controlling actions of the microcontroller circuit.

11. The medical apparatus of claim 1, and further comprising a display device, coupled to the microcontroller circuit, receiving and displaying the ECG and EEG signals.

12. The medical apparatus of claim 11, wherein the display device is a liquid crystal display (LCD).

13. The medical apparatus of claim 1, wherein the EEG detecting circuit further comprising:
an electrode module, attached to and detecting the human head, and producing the EEG signal;
a first amplification circuit, receiving and amplifying the EEG signal;
a filter circuit, receiving the EEG signal amplified by the first amplification circuit, and filtering the EEG signal; and
a second amplification circuit, receiving the EEG signal filtered by the filer circuit, and amplifying the EEG signal.

14. The medical apparatus of claim 13, wherein the electrode module includes six electrodes.

15. The medical apparatus of claim 13, wherein the filter circuit further include:
a high-pass filter, filtering the low-frequency component of the EEG signal;
a low-frequency filter, filtering the high-frequency component of the EEG signal filtered by the high-pass filter;
a band-reject filter, filtering the noise frequency of the EEG signal filtered by the low-pass filter.

16. The medical apparatus of claim 15, wherein the frequency of the noise frequency is 60 Hz.

17. The medical apparatus of claim 1, wherein the ECG detecting circuit further includes:
an electrode module, attached to and detecting the human heart, and producing the ECG signal;
a first amplification circuit, receiving and amplifying the ECG signal;
a filter circuit, receiving the ECG signal amplified by the first amplification circuit, and filtering the ECG signal; and
a second amplification circuit, receiving the ECG signal filtered by the filer circuit, and amplifying the ECG signal.

18. The medical apparatus of claim 17, wherein the filter circuit further include:
a high-pass filter, filtering the low-frequency component of the ECG signal;
a low-frequency filter, filtering the high-frequency component of the ECG signal filtered by the high-pass filter;
a band-reject filter, filtering the noise frequency of the ECG signal filtered by the low-pass filter.

19. The medical apparatus of claim 18, wherein the frequency of the noise frequency is 60 Hz.

20. The medical apparatus of claim 1, wherein the storage unit is a flash memory.
